(19) United States
(12) Patent Application Publication
(10) Pub. No.: US 2016/0262731 A1
(43) Pub. Date: Sep. 15, 2016

(54) NERVE STIMULATION BIOPSY DEVICE

(71) Applicants: Michel Kliot, Chicago, IL (US); John Bode, Wauwatosa, WI (US); Michael Heiferman, Chicago, IL (US); Ryan Khanna, Chicago, IL (US); Yunli Ma, Evanston, IL (US); Komal Prem, Wadsworth, IL (US); Nick Messana, Chicago, IL (US); Colin Russi, Chicago, IL (US)

(72) Inventors: Michel Kliot, Chicago, IL (US); John Bode, Wauwatosa, WI (US); Michael Heiferman, Chicago, IL (US); Ryan Khanna, Chicago, IL (US); Yunli Ma, Evanston, IL (US); Komal Prem, Wadsworth, IL (US); Nick Messana, Chicago, IL (US); Colin Russi, Chicago, IL (US)

(21) Appl. No.: 15/064,907

(22) Filed: Mar. 9, 2016

Related U.S. Application Data
(60) Provisional application No. 62/130,989, filed on Mar. 10, 2015.

(51) Int. Cl. A61B 10/02; A61B 5/04

(52) U.S. Cl. CPC A61B 10/0233; A61B 5/04001

(57) ABSTRACT

A nerve stimulation biopsy device configured to deliver a precise and adjustable stimulus signal into a biopsy site. The stimulus signal is used to distinguish between functioning nerve fibers and target tissue. Also disclosed is a method for safely performing a biopsy procedure on a biopsy site of a patient, which utilizes introducing a stimulus signal to the biopsy site.

### CROSS-REFERENCE TO RELATED APPLICATION

This application claims the benefit of U.S. Provisional Patent Application Ser. No. 62/130,989, filed Mar. 10, 2015, the disclosure of which is hereby incorporated by reference in its entirety.

### BACKGROUND OF THE INVENTION

1. Field of the Invention

The present subject matter relates generally to a nerve stimulation biopsy device. More specifically, the present invention relates to a biopsy device possessing a tip configured to deliver an electrical stimulus to distinguish between functioning nerve fibers and target tissue, such as tumorous tissue.

2. Discussion of the Prior Art

Surgery is being performed more rapidly and safely, through smaller incisions, using a variety of technologies, including intraoperative imaging and endoscopic instrumentation. Pathology frequently involves peripheral nerves, often embedded in deep and difficult to access locations.

Currently, biopsies of nerve and associated pathology, such as nerve tumors (e.g., Neurofibromas and Schwannomas), are performed using a biopsy device that cannot identify or distinguish functioning nerve fibers from pathological nerve or tumor tissue. As a result, biopsies often damage functioning nerve tissue, resulting in long lasting pain and even loss and/or altered sensory and/or motor function. Also, in many cases, physicians will elect to fully remove the tumor in surgery, under general anesthesia, before performing a biopsy rather than risk the negative patient outcomes associated with the biopsy procedure. This presents additional risks to the patient and is often unnecessary as these tumors are rarely malignant.

While there is no device in the market that addresses the specific need in any way, any improvement will compete with the current state of the art technology. For example, while percutaneous image guided biopsy devices using ultrasound, CT, or MRI, assist in guiding the path for incision, they cannot distinguish between functioning nerve fibers and pathological tissues.

Current guided methods have disadvantages. An MRI guided biopsy is an expensive and lengthy procedure. Its duration depends on factors such as: (a) the availability of the MRI suite occupation time, (b) number of operators, (c) number of image acquisitions, and (d) times the patient was moved in and out of the closed magnet. An ultrasound guided biopsy has a number of deficiencies, such as attenuation of the sound beam, and obstruction of visualization. CT guided biopsies expose patients and surgeons to radiation and may be especially harmful for women and children.

A structural improvement to the full core biopsy device with a secondary cutting cannula can be found in a vibration assisted device. In a vibration assisted biopsy device, vibrations ease the advancement of the cannula through tissue to the site of interest, decreasing damage to the tissue, and relieving pain. However, because the vibration assisted biopsy device cannot distinguish between functioning nerve fibers and tumorous tissue, the nerve fibers are still at risk.

### SUMMARY

The present disclosure provides an advantageous biopsy device that decreases the likelihood of damage to functioning nerve fibers by assisting the user in distinguishing between nerve fibers and tumorous tissue. More particularly, this disclosure provides a nerve stimulation biopsy device whose tip is configured to deliver a precise electrical stimulus (stimulus amplitude, frequency and duration could be controlled) to help localize the cannula in relation to nerve tissue and prevent damage to functioning nerve fibers.

The biopsy device in accordance with the present invention has the following advantages. First, the nerve stimulation biopsy device has a tip that is capable of delivering a precise electrical stimulus to distinguish between functioning and tumorous nerves, and to prevent damage to functioning nerve fibers. Second, the stimulation signal emitted is capable of being adjusted and controlled in accordance with patient responses, allowing the user to determine the exact proximity to healthy nerve tissue.

In a first aspect, this disclosure provides a nerve stimulation biopsy device, comprising a cannula assembly including an outer cannula having a proximal end and a distal end and a middle section having a substantially hollow cross-section between said proximal and distal ends, wherein said cannula assembly is configured to deliver a stimulus signal through said distal end. The device further includes a handle connected to the cannula assembly, an electrical power source connected to the handle, and a control member connected to the handle and being in communication with a controller, wherein said controller is configured to deliver an electrical amperage to the inner surface of the outer cannula.

In another aspect, this disclosure provides a method for safely performing a biopsy procedure on a biopsy site of a patient. The method includes inserting a distal end of the cannula assembly into the biopsy site, and introducing via the cannula assembly a stimulus signal of a predetermined strength into the biopsy site. The method further includes receiving feedback from the patient and removing the distal end of the cannula assembly from the biopsy site if said feedback is associated with a positive physical response or continuing with the procedure if said feedback is associated with a negative physical response, wherein said positive physical response is characterized by pain, sensation or muscle reflex and said negative physical response is characterized by an absence of pain, sensation and muscle reflex, and removing the target tissue from the biopsy site

As above noted, the example nerve stimulation biopsy devices and example methods of using the same of this disclosure provide several advantageous features. It also is to be understood that both the foregoing general description and the following detailed description are exemplary and provided for purposes of explanation only, and are not restrictive of the subject matter claimed. Further features and objects of the present disclosure will become more fully apparent in the following description of the preferred embodiments and from the appended claims.

### BRIEF DESCRIPTION OF THE DRAWINGS

In describing the preferred embodiments, reference is made to the accompanying drawing figures wherein like parts have like reference numerals, and wherein:

FIG. 1 is a top plan view of an example nerve stimulation biopsy device in accordance with the present disclosure;

FIG. 2 is a side plan view of the example nerve stimulation biopsy device of FIG. 1; and

FIG. 3 is a front plan view of the example nerve stimulation biopsy device of FIG. 1.

It should be understood that the drawings are not to scale. While some mechanical details of example nerve stimulation biopsy devices, including other plan and section views of the example shown and of examples that may have alternative configurations, have not been included, such details are considered well within the comprehension of those of skill in the art in light of the present disclosure. It also should be understood that the present invention is not limited to the example embodiments illustrated.

### DETAILED DESCRIPTION OF THE PREFERRED EMBODIMENT

For the following defined terms, these definitions shall be applied, unless a different definition is given in the claims or elsewhere in this disclosure. As used in this disclosure and the appended claims, the singular forms “a”, “an”, and “the” include plural referents unless the content clearly dictates otherwise. As used in this disclosure and the appended claims, the term “or” is generally employed in its sense including “and/or” unless the content clearly dictates otherwise.

While the present invention will be described in the specific context of a peripheral nerve biopsy device, the invention is not limited to just peripheral nerve biopsy instruments. The present invention may be used in conjunction with a variety of medical or other instruments and is effective for use in not only peripheral nerve biopsy retrieval procedures, but also with other instruments to perform other tissue removal procedures, or other procedures and associated instruments as would be appreciated by those of ordinary skill in the art, in light of the present disclosure.

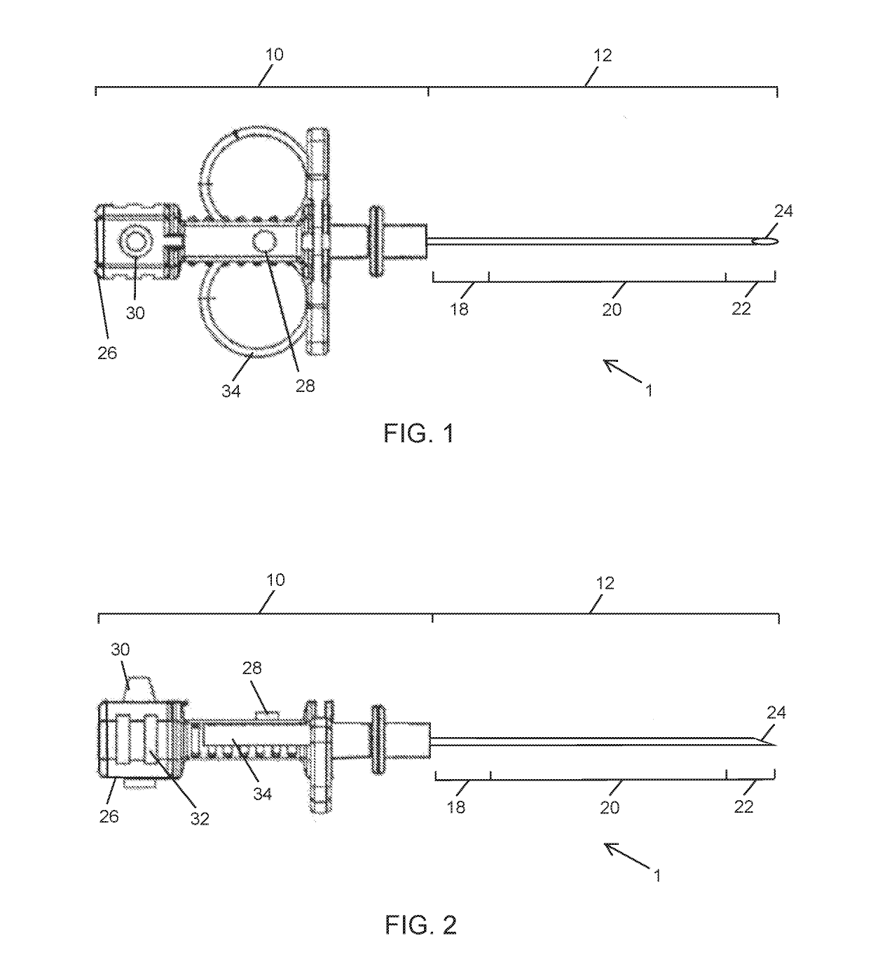

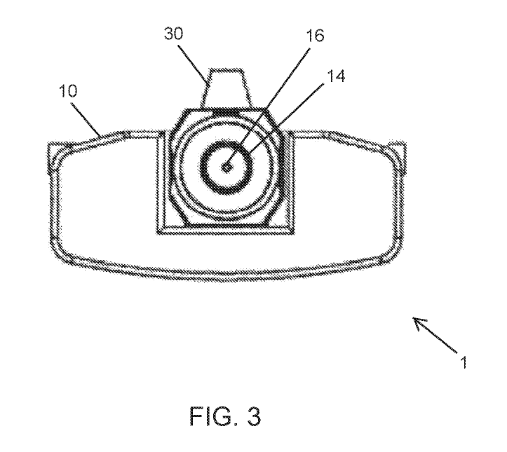

An example peripheral nerve biopsy device 1 is shown in FIGS. 1-3, comprising a handle assembly 10 structurally and operably connected to a cannula assembly 12. The cannula assembly 12 is used to penetrate a target tissue mass, such as of tumorous tissue, located within the peripheral nerve sampling site for removal of a biopsy sample. The handle assembly 10 is configured to control the acquisition and removal of the biopsy sample from the peripheral nerve sampling site. As described and illustrated herein, the handle assembly 10 is further configured to control the introduction of a stimulus signal, through the cannula assembly 12, into the peripheral nerve sampling site to differentiate healthy nerve tissue from target tissue.

The cannula assembly 12, shown in FIGS. 1-3, comprises an outer cannula 14 having a cylindrical tube shape, and housing an inner member 16. The outer cannula 14 is divided into a proximal end section 18, a middle section 20, and a distal end section 22, with the middle section 20 having a substantially hollow cross section between the proximal end 18 and distal end 22. The distal end section 22 is shown in FIGS. 1 and 2 as having a beveled, pointed configuration 24, with sharp edges. Such a configuration facilitates cutting through soft tissue, thus facilitating entry of the cannula assembly 12 of the peripheral nerve biopsy device 1 into a peripheral nerve sampling site. However, it is contemplated that the distal end section may be of various lengths and other distal end section configurations, such as a saddle tip, or other desired configuration, may be used with the present invention, depending on the specific biopsy application.

The external portion of the proximal end section 18 and middle section 20 are coated in an insulating material, while the distal end section 22 is not similarly coated. The extent to which the coating may extend toward the distal tip may depend on the type of tissue to be biopsied, the proximity to other tissues or other factors. As will be described herein, this arrangement facilitates the precise introduction of a stimulus signal via the distal end section 22 into the peripheral nerve biopsy site. The insulating material may be comprised of a vinyl coating. However, it is contemplated that other insulating, non-conductive materials that are well known in the art may be used.

The inner member 16 of the example cannula assembly 12 comprises a sharpened stylet used for removing target tissue from the peripheral nerve sampling site. The inner member 16 may comprise a hollow inner cannula, an ejector rod for forcing tissue from the outer cannula 14, as well as other types of inner member structures depending on the specific biopsy application.

The handle assembly 10, shown in FIGS. 1 and 2, is connected to and operably associated with the proximal end section 18 of the cannula assembly 12. The handle assembly 10 comprises an electrical power source 26, a first control member 28 operably associated with the inner member 16, and a second control member 30 operably associated with a controller 32 and the power source 26. An example embodiment of the handle assembly 10 is shown in the drawings as having an ergonomic design, with the power source 26, first control member 28, second control member 30 and finger rings 34 arranged to facilitate easy, comfortable use. However, it will be appreciated that the precise configuration of these elements may vary, as desired for a specific implementation. In addition, a tripod or other needle stabilization device may be configured for engagement with the cannula assembly and/or the handle assembly, so as to assist the user in locating and steadying the device during use.

The first control member 28 is configured to effect movement of the inner member 16 to effect the taking of a biopsy sample, in response to a user input. The first control member 28 may be a button, plunger, or other configuration, as desired, depending on the specific biopsy application.

The second control member 30 is in communication with a controller 32 housed within the handle assembly 10. The second control member 30 communicates user input regarding the desired amperage of a precise electrical stimulus signal to the controller 32. The second control member 30 may be a dial, digital input, or other type of structure, to convey the user input.

The controller 32 is in communication with the power source 26. The controller 32 relays the desired amperage from the power source 26, which is configured to apply a stimulus signal of the desired amperage to the inside of the outer cannula 14. The stimulus signal conducts along the inside of the insulated proximal end section 18 and middle section 20 until it reaches the exposed distal end section 22 and emits into the peripheral nerve biopsy site.

The operation of the peripheral nerve biopsy device 1 will now be described in further detail. The biopsy procedure is initiated by inserting the cannula assembly 12 into the peripheral nerve biopsy site by manual or automated means. As previously mentioned a stabilization device, such as a tripod, may be utilized when inserting the cannula assembly. With the example device 1 shown, the user preferably grasps the handle assembly 10 and inserts the cannula assembly 12 into the peripheral nerve biopsy site, distal end section 22 first. An imaging system preferably is used to guide the positioning of the cannula assembly 12. Generally, the cannula assembly 12 is positioned within the peripheral nerve biopsy site such that, when the biopsy sample is taken, at least part of the target tissue is included in the sample.

Any suitable imaging system may be used to guide the cannula assembly 12 to the peripheral nerve biopsy site, for example radiography, ultrasound, or MRI. The outer cannula 14, distal end section 22, or inner member 16 may be made from one or more materials, and/or shaped or provided with markings to enhance the visibility of the structures with a corresponding imaging system.

After the initial positioning of the cannula assembly 12, the user introduces a stimulus signal of a predetermined amperage into the peripheral nerve biopsy site by way of the distal end section 22. The stimulus signal may be turned on and off and adjusted, though not generally exceeding 5 mA, by user inputs to the second control member 30. If the distal end section 22 is in close proximity to healthy nerve tissue, the stimulus will result in a physical response felt by the patient. The user receives feedback from the patient and may remove the distal end section 22 of the cannula assembly 12 from the biopsy site if the feedback is associated with a positive physical response or continuing with the procedure if the feedback is associated with a negative physical response, wherein the positive physical response is characterized by pain, sensation or muscle reflex and the negative physical response is characterized by an absence of pain, sensation and muscle reflex. The user may determine the exact distance to healthy nerve tissue by adjusting the strength of the electrical stimulus signal, as a stronger signal will stimulate nerves at a greater distance than a weaker signal. The user may proceed with the biopsy procedure once it is determined that the distal end section 22 is not in close proximity to healthy nerve tissue. The user may receive the feedback, whether a positive or negative physical response, via communication with the patient or observation.

After the cannula assembly 12 is determined to have been safely positioned, the user activates the first control member 28 causing the inner member 16 to extend into the target tissue and retrieve a biopsy sample. The cannula assembly 12 is then removed from the nerve biopsy site.

While this invention is susceptible of embodiment in many different forms, there is shown in the drawings one specific embodiment with the understanding that the present disclosure can be considered as an exemplification of the principals of the invention and is not intended to limit the invention to the example embodiment illustrated, and is only limited by the appended claims and legal equivalents thereof.

1. A nerve stimulation biopsy device, comprising:
a cannula assembly including an outer cannula having a proximal end and a distal end and a middle section having a substantially hollow cross-section between said proximal and distal ends, wherein said cannula assembly is configured to deliver a stimulus signal through said distal end;
a handle connected to the cannula assembly;
an electrical power source connected to the handle; and
a control member connected to the handle and being in communication with a controller, wherein said controller is configured to deliver an electrical amperage to the inner surface of the outer cannula.

2. The nerve stimulation biopsy device according to claim 1, wherein said controller is configured to receive user input to control the electrical amperage.

3. The nerve stimulation biopsy device according to claim 1, wherein said user input includes a frequency selection, and electrical amperage is associated with the selected frequency.

4. The nerve stimulation biopsy device according to claim 1, wherein said outer cannula has a beveled tip at the distal end.

5. The nerve stimulation biopsy device according to claim 1, wherein said cannula has a saddle tip at the distal end.

6. The nerve stimulation biopsy device according to claim 1, wherein said power source is a battery.

7. The nerve stimulation biopsy device according to claim 1, wherein the external portion of said outer cannula, from the start of the proximal end up to the start of the distal end, is covered by a coating of a non-conductive material.

8. The nerve stimulation biopsy device according to claim 7, wherein said non-conductive material is vinyl.

9. The nerve stimulation biopsy device according to claim 1, further comprising an inner member housed within the outer cannula.

10. The nerve stimulation biopsy device according to claim 9, wherein said inner member is a sharpened stylet.

11. The nerve stimulation biopsy device according to claim 9, wherein said inner member is a hollow inner cannula.

12. A method for safely performing a biopsy procedure on a biopsy site of a patient, the method comprising;
inserting a distal end of the cannula assembly into the biopsy site;
introducing via the cannula assembly a stimulus signal of a predetermined strength into the biopsy site;
receiving feedback from the patient and removing the distal end of the cannula assembly from the biopsy site if said feedback is associated with a positive physical response or continuing with the procedure if said feedback is associated with a negative physical response, wherein said positive physical response is characterized by pain, sensation or muscle reflex and said negative physical response is characterized by an absence of pain, sensation and muscle reflex; and
removing the target tissue from the biopsy site.

13. The method according to claim 12, further comprising adjusting the strength of the stimulus signal to determine the proximity of the distal end of the cannula assembly to healthy nerve fibers at or near the biopsy site.

14. The method according to claim 12, wherein the stimulus signal further comprises an electrical stimulus signal having a selected amperage.

15. The method according to claim 12, wherein introducing a stimulus signal of a predetermined strength includes selecting a frequency, and wherein the stimulus signal includes an electrical amperage that is associated with the selected frequency.
